(19) United States
(12) Patent Application Publication
(10) Pub. No.: US 2006/0045356 A1
(43) Pub. Date: Mar. 2, 2006

(54) Characterisation of paper

(76) Inventors: Markus Turtinen, Oulu (FI); Olli Silven, Oulu (FI); Matti Pietikainen, Oulu (FI); Matti Niskanen, Oulu (FI); Topi Maenpaa, Oulu (FI)

Correspondence Address: OSTROLENK FABER GERB & SOFFEN, 1180 AVENUE OF THE AMERICAS, NEW YORK, NY, 100368403, US

(21) Appl. No.: 10/526,831

(22) PCT Filed: Aug. 27, 2003
(86) PCT No.: PCT/FI03/00626

§ 371 Date: Jul. 20, 2005

(30) Foreign Application Priority Data
Sep. 3, 2002 (FI) 20021578

(51) Int. Cl. G06T 7/40; G06N 3/02; G01N 33/34; D21F 11/00

(52) U.S. Cl. 382/225; 162/198; 162/263; 382/141; 382/159

(57) ABSTRACT

A method and system for characterising paper, where from images of numerous paper samples are extracted multi-dimensional features describing features of the paper; the said features are entered as input into a learning classifier operating in an unsupervised manner, which produces an projection of the said data of each picture part in a low-dimension space in such a way that paper grades having close properties produce close projection in the low-dimension space and the classification results depicted in the low-dimension space are used to aid classification.

The invention relates to the characterisation and classification of paper quality by using computer vision or other two-dimensionally descriptive method.

To the application is appended a bibliography, which is referred to by reference numerals in square brackets. Prior art is referred to in the form of cited references in connection with the aspect at hand, respectively.

The aim of the invention is to accomplish a method for the characterisation of paper quality that will provide more reliable classification than current methods, without variation due to human factors.

Paper grading systems based on computer vision—which represent the prior art—were previously founded on supervised learning methods and old and inefficient features computed from images. As features have usually been used measurements obtained from co-occurrence matrices, power spectrum analysis and the specific perimeter feature. Also, the average of the grey shades and variance of the images have been presumed to represent variations in paper grammage. Of the features has been formed a numerical quantity, which describes the quality of paper. On the basis of this numerical quantity, the formation or other properties of the paper have then been classified. [1, 2, 3, 4, 5]

The old textural features are unable to provide very accurate information on paper texture and they are sensitive to changes in conditions, such as lighting. When poorly discriminating features are combined with supervised training of a classifier, the characterisation capacity of the system is further impaired. This is due to the fact that the conventional supervised methods are extremely sensitive to human errors. People usually make errors in selecting the training samples and in naming them. In addition, the selections made by humans are subjective and thus the interpretations of different people differ from one another. From the point of view of quality inspection this is undesirable. Re-training a system based on supervised learning methods is difficult, should the changes in conditions so require. This is often the case, because less developed textural features are extremely sensitive to changes in the conditions.

A problem has been that paper has been analysed with poorly discriminating textural features. Furthermore, attempts have been made to specify class boundaries in an already fragmented and non-normally distributed feature space by means of parametric methods. Supervised methods have been used in training the classifiers and in seeking the class boundaries, which increases the amount of errors.

In characterising paper, the aim is to classify papers sharing the same properties in the same category. Paper may be imaged throughout its manufacture, which will also give information on the properties of good or poor paper during the different stages of manufacture. Without characterisation, on the basis of images alone, it is not possible to seek useful information on the process, because the assessment and classification of images is very difficult for man as well as being subjective and, in addition, processing a large amount of data without automatic classification based on numerical values or symbols is impossible. By means of characterisation, the quality of paper can be classified into several classes on the basis of which the operation of the manufacturing process can be traced and attempts can be made to improve certain properties of the paper, so long as it is known which factors affect the quality of paper, and what the paper has been like at each stage of manufacture, respectively. Characterisation itself does not have to take a stand on the quality of the paper, it suffices that similar papers are classified into the same class. The process may be controlled or the paper can be classified into quality classes in accordance with the classification.

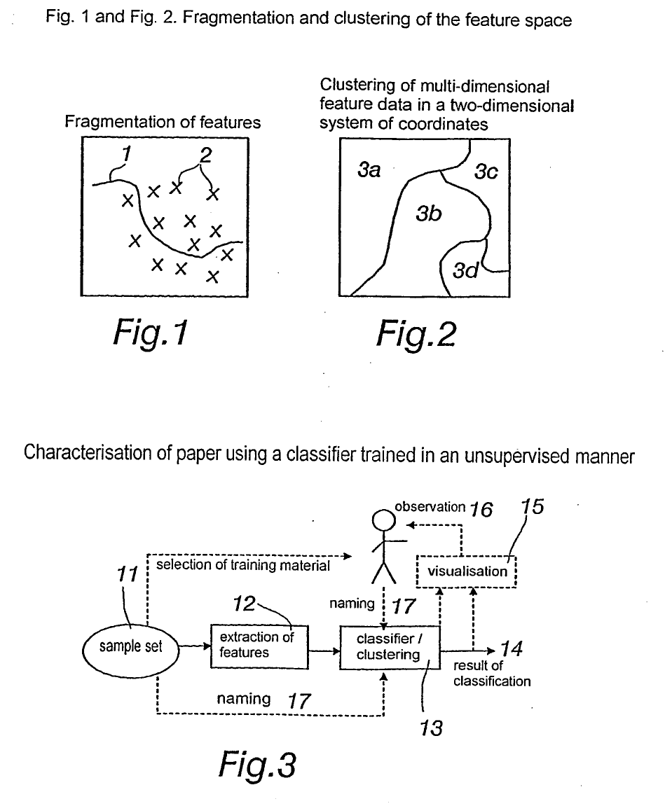

In computer vision methods, the aim is to calculate a number of features, which will describe the properties of paper as accurately as possible [1, 2, 3, 4, 5]. Typical properties are, for example, the printability and tensile strength of the paper. The features calculated are numerical quantities and they form clusters fragmented in a multi-dimensional feature space. The feature space may be extremely multi-dimensional, and it is obvious that the features describing different paper grades are difficult to find in the fragmented space. FIG. 1 shows an example of a feature space presented, for the sake of simplicity, in a two-dimensional system of coordinates. The crosses in the Figure represent the values of the features, and the line drawn in the Figure the possible change in the printability properties of the paper.

The specification refers to the following Figures:

FIG. 1 shows the fragmentation of features and the boundary of properties.

FIG. 2 shows the clustering of multi-dimensional feature data in a two-dimensional system of coordinates.

FIG. 3 shows a diagram in principle of classification according to the invention.

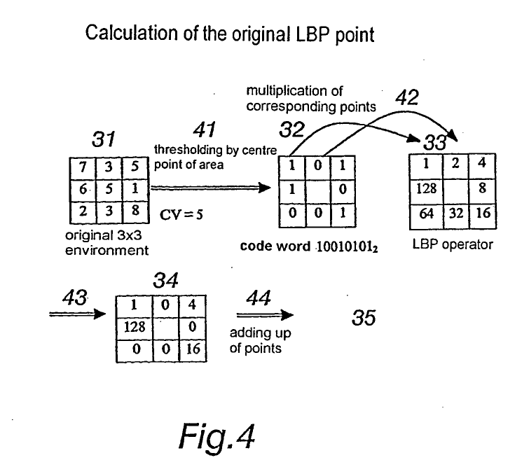

FIG. 4 shows the calculation of a 3×3 size LBP feature.

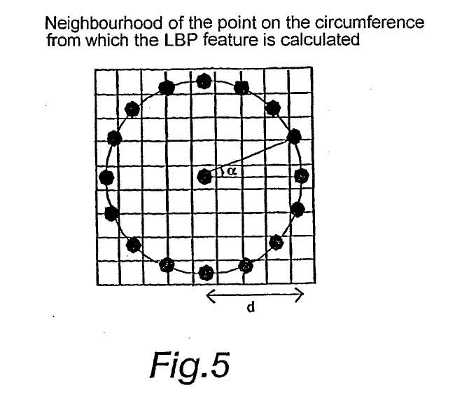

FIG. 5 shows the neighbourhood of a point on the circumference from which the LBP feature is calculated.

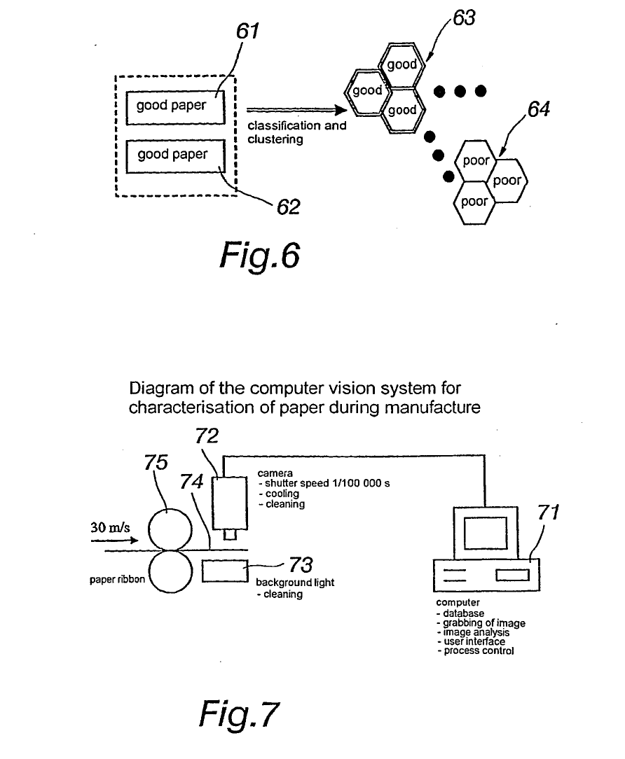

FIG. 6 shows the use of a SOM as a classifier.

FIG. 7 shows a diagrammatic view of paper characterisation during manufacture.

Conventional parametric methods are unable to find the boundaries between different paper grades accurately, because they make assumptions on the distribution of data. In the method according to the invention, the data is first depicted in a two-dimensional system of coordinates. Each cluster is given a label on the basis of the type of paper the cluster represents. In other words, deductions on the quality of the paper can be made on the basis of the location of the sample in the two-dimensional system of coordinates. FIG. 2 shows an example of describing a multi-dimensional feature space in a two-dimensional system of coordinates by means of a method, which maintains the local structure of the data and the mutual distances between samples [6, 7, 8, 9, 10]. Labels 3a-3d represent different properties of the paper; paper classified in an area marked by the same label is similar to other papers in the same class with respect to the property in question. The labels are given afterwards and, for example, tensile strength, degree of gloss or printability are usually divided into different regions and obviously have different labels.

In the method, the data is organised automatically in such a way that the mutual locations of the samples in the new system of coordinates are the same as in the original multi-dimensional feature space. Reliable deductions on paper grades can be made on the basis of where they are located in the new system of coordinates. At first, no deductions whatsoever are made on the distribution of the data, and it may be of any kind. Papers having different textures may still have similar print properties. This may be taken into account when labelling the different clusters. With efficient textural features, such as LBP, the surface texture of paper can be analysed extremely efficiently [11, 12].

In the present invention, an unsupervised learning method, efficient grey-shade variant textural features and illustrative visualisation of multi-dimensional feature data are combined by reducing the dimensions of the feature space. In the method, human assumptions and deductions do not need to be made concerning the training material, but the training data will be organised automatically in accordance with its properties. The multi-dimensional feature space is depicted in an illustrative form and the location of the samples in the feature space can be visualised.

New, sophisticated texture methods give precise information on the microstructure of the texture. Such grey-shade invariant textural features are, for example, LBP features, which measure local binary patterns, and its modifications [11, 12]. When the surface of paper is examined using these features, important properties of the paper may be discovered. By combining efficient textural features with an unsupervised learning method, the accuracy of grading can be greatly improved.

A diagrammatic view of the method is shown in FIG. 3. From the training set 11 are first calculated textural features at stage 12, which are then used to train the classifier. The dimensions of the multi-dimensional feature space are reduced in order that it can be illustratively visualised. Classification is also carried out by using a new feature space 14. The task remaining to man is to name and select classified areas and, at the next stage, to render them into a more easily understandable form or to place the paper grades in an order of superiority, so that the process may subsequently be regulated on the basis of them. It is also a task for man to select the training set in such a way that a representative sample of different papers is obtained. These tasks are indicated by reference numerals 15, 16, 17 and 18.

In the method, the properties of paper are first described by means of efficient textural features, which reduces the fragmentation of the feature space markedly. A multi-dimensional feature space is depicted in a low-dimension system of coordinates in such a way that the local structure of the data is preserved. The clusters in the low-dimension system of coordinates represent different paper grades. The different clusters are named in accordance with the paper grade represented by the cluster in question. After this, in the new system of coordinates can be classified different grades of paper by finding the cluster to which the paper being examined is clustered. A diagram representing a clustered feature space is shown in FIG. 2.

The features may be extracted, for example, by using textural quantities based on local binary patterns. LBP (Local Binary Pattern) features describe patterns appearing in a local image-level environment [11, 12]. An original LBP feature [11] is, for example, a textural feature calculated from a 3×3 environment, the calculation of which is illustrated in FIG. 4. In the example shown in the Figure, the 3×3 environment 31 is categorised by threshold values (arrow 41) in accordance with the grey shade of the centre point (CV) of the environment so as to have two levels 32: pixels greater than or equal to the threshold value CV are given the value 1, and lower values obtain the threshold value 0. Subsequent to categorisation by threshold values, the values 32 obtained are multiplied (arrow 42) by an LBP operator 33, which gives an input matrix 34, the elements in which are added up (arrow 44), which gives the value of the LBP. Another way of conceiving the calculation of the LBP is to form an 8-bit code word directly from the threshold value environment. In the case of the example, the code word would be 10010101₂, which is 149 in the decimal system.

Of LBP features have also been created various multi-resolution and rotation invariant methods [12]. In addition, the effect of different binary patterns on the performance of the LBP operator have been examined, whereby it has been made possible to omit certain patterns in forming the feature distribution [12]. In this way it has been possible to shorten the LBP feature distribution.

Multi-resolution LBP means that the neighbourhood of the point has been selected from several different distances. The distance may in principle be any positive number, and the number of points used in the calculation may also vary according to distance. FIG. 5 shows the neighbourhood of a point at a distance of four (d=4). Around the point is drawn a circle, the radius of which is equal to the distance selected. From the circumference are selected samples at distances indicated by the angle α in such a way that Nα=2π, where N is the number of selected samples. If a sample on the circumference does not match a pixel accurately, it is interpolated, by means of which the coordinates of the point are made to correspond to the coordinates on the circumference. Distances typically used are 1, 2 and 3, and the numbers of samples are correspondingly 8, 16 and 24. The more points are selected, the greater the LBP distribution obtained. A 24-dimensional feature space produces a LBP distribution containing over 16 million poles.

Using extensive LBP distributions in calculation is cumbersome. The size of the distribution can be reduced to a more reasonable size for calculation by taking into account only a certain, pre-selected part of the LBP codes. The selected codes are so-called continuous binary codes in which the numbers on the circumference include at most two bit exchanges from 0 to 1 or vice versa. Thus the code words selected contain long, continuous chains comprised of zeros and ones. The selection of the codes is based on the knowledge that by means of certain LBP patterns can be expressed as much as over 90% of the patterning in the texture. By using only these continuous binary chains in calculation, an LBP distribution of 8 samples can be reduced from 256 to 58. An LBP distribution with 16 samples is, on the other hand, reduced from over 65 thousand to 242, and a distribution of 24 samples from over 16 million to 554 [12].

In the calculation of the LBP feature of a rotation invariant is included a pre-selected subset of LBP patterns [12]. The patterns have been selected in such a way that they are invariant to rotation taking place in the texture. Using the LBP features of rotation invariants in a non-invariant problem reduces the capacity of the feature. The characterisation of paper is not, however, a rotation invariant problem.

Classification and clustering may be carried out, for example, by applying techniques based on self-organising maps [13]. A self-organising map, the SOM, is a method of unsupervised learning based on artificial neural networks. The SOM makes possible the presentation of multi-dimensional data to man in a more illustrative, usually two-dimensional form.

A SOM aims to present data in such a way that the distances between samples in the new two-dimensional system of coordinates will correspond as accurately as possible to the distances between the real samples in their original system of coordinates. The SOM does not aim to separately search the data for the clusters it may contain or to display them, but instead presents an estimate of the probability density of data as reliably as possible, while maintaining its local structure. This means that if the two-dimensional map shows dense clusters formed by samples, then these samples are located close to one another in the feature space also in reality [13].

In order that the SOM can be used to group a certain type of data, it must first be trained. The SOM is trained by means of an iterative, unsupervised method [13]. Following the training of the SOM, there is a point set in the multi-dimensional space for each node on the map, to which the node corresponds. An algorithm has adjusted the map by means of training samples. Multi-dimensional vectors form a non-linear projection in the two-dimensional system of coordinates, thus making clear visualisation of the clusters possible [13].

The use of the SOM as a classifier is based on the clustering of similar samples close to one another, which means that they can be defined as their own classes on the map. The samples of nodes far from each other are mutually different, whereby they can be distinguished to belong to different classes. FIG. 6 shows the clustering of good and poor paper in opposite corners of the map. FIG. 6 shows the use of the SOM as a classifier. Samples 61, 62 in the Figure are classified in classes 63, 64. As a rough example has been shown the classification of good paper 61 in class area 63, and the classification of poor paper in area 64. It should be noted that there may be several areas of both good and poor paper fragmented in different parts of, for example, a two-dimensional space, but in such a way, however, that for example all paper classified in area 64 is poor in the same respect. It is understandable, that it is very useful for the paper manufacturer to know which conditions produce paper of the said kind, so that the conditions producing poor quality can be avoided in manufacture. This is possible by monitoring the production parameters and by continuously classifying the quality of paper, whereby new aspects will be learnt of the operation of the process. It is also possible to enter the process parameters and the results of paper classification into another SOM classifier, whereby a system learning from errors is obtained, which can be used as an aid in process control. This will give as a final outcome a classification which describes the conditions of manufacture with respect to the quality of paper. The system thus learns, for example the effect of hundreds of variables on paper quality.

Above is described classification according to the invention using SOM classification, but any unsupervised clustering method is suitable for use in the classification according to the invention, for example, the LLE, ISOMAP and GTM techniques which are not actual neural network techniques.

The method is suitable for use in the quality inspection of paper during paper manufacture, for example, as shown in diagram 7. Pictures are taken with a fast camera of the moving paper web 74 in connection with the paper machine 75. The diagram in the Figure shows a background light 73; depending on the need also, for example, a diagonal front light can be used. After this, deductions on the qualitative properties of the paper being produced can be made, and the any adjustments in the progressing of the process may be carried out. The method being presented here would be used in connection with the computer 71 shown in the Figure. Rapid image analysis and an illustrative user interface for extensive measurement data provide an enormous amount of additional information on the paper being produced to the paper manufacturers themselves.

Features are extracted from the pictures taken during the image analysis by means of the techniques mentioned above, and classification into different quality classes is carried out. By means of the user interface, the progressing of the quality of the paper can be followed as production progresses.

By means of the method, paper can be analysed almost throughout its production cycle. The power of the background light must, however, be increased if pictures are taken of already coated paper. In addition, the capacity of textural features may be impaired with coated papers.

Exact information on the quality of paper during its production facilitates studies carried out by the paper manufacturer. An automation manufacturer may integrate the system to be a part of the overall process and its adjustment.

The invention is characterised by what is presented in the independent claims and the dependent claims describe its preferred embodiments.

### APPENDIX: BIBLIOGRAPHY

[1] Cresson T. M., Tomimasu H. & Luner P. (1990) Characterization of Paper Formation, Part 1: Sensing Paper Formation. Tappi Journal: Vol. 73, No. 7: p. 153-159.
[2] Cresson T. & Luner P. (1990) Characterization of Paper Formation, Part 2: The Texture Analysis of Paper Formation. Tappi Journal: Vol. 73, No. 12: p. 175-184.
[2] Cresson T. & Luner P. (1991) Characterization of Paper Formation, Part 3: The Use of Texture Maps to Describe Paper Formation. Tappi Journal: Vol. 74, No. 2: p. 167-175.
[3] Sudhakara R. P., Stridhar R., Gopal A., Meenakshi K., Revathy R., Chitra K. & Palaniandi D. (2001) Optical Paper Formation Analyzer. CEERI Centre, India.
[4] Bernie J. P. & Douglas W. J. M. (1996) Local Grammage Distribution and Formation of Paper by Light Transmission Image Analysis. Tappi Journal: Vol. 79, No. 1: p. 193-202.
[5] Bouyndain M., Colom J. F., Navarro R. & Pladellorens J. (2001) Determination of Paper Formation by Fourier Analysis of Light Transmission Images. Appita Journal: Vol. 54, No. 2: p. 103-105, 115.
[6] Kohonen T. (1997) Self-organizing Maps. Springer-Verlag, Berlin, Saksa, 426 p.
[7] Roweis S. T. & Saul L. K. (2000) Nonlinear Dimensionality Reduction by Locally Linear Embedding. Science Magazine, Vol 290, 22 Dec. 2000: p. 2323-2326.
[8] Roweis S. T. & Saul L. K. (2001) An Introduction to Locally Linear Embedding. URL: http://www.cs.toronto.edu/˜roweis/lle/papers/lleintroa4.pdf (13.5.2002).
[9] Svensên J. F. M. (1998) GTM: The Generative Topographic Mapping. Doctoral thesis. Aston University, Englanti, 108 p.
[10] Tenenbaum J. B. (1998) Mapping a Manifold of Perceptual Observations. Advances in Neural Information Processing Systems, Vol. 10.
[11] Ojala T., Pietikäinen M. & Harwood D. (1996) A Comparative Study of Texture Measures With Classification Based on Feature Distributions. Pattern Recognition, Vol. 29, No. 1, p. 51-59.
[12] Ojala T., Pietikäinen M. & Mäenpää T. (2002) Multiresolution Gray-Scale and Rotation Invariant Texture Classification with Local Binary Patterns. IEEE Transactions on Pattern Analysis and Machine Intelligence, Vol. 24, No. 7.
[13] Kohonen T. (1997) Self-organizing Maps. Springer-Verlag, Berlin, Saksa, 426 p.

1. A method for characterising features of paper based on computer vision, characterised in that from pictures of numerous paper samples are extracted multi-dimensional features describing features of paper; the said features are entered as input into a learning classifier operating in an unsupervised manner, which produces a projection of the said data of each picture part in a low-dimension space, so that paper grades having close properties produce close projections in the low-dimension space and the classification results projected in the low-dimension space are used to aid classification.

2. A method for characterising paper as claimed in claim 1, characterised in that the said learning system operating in an unsupervised manner is an unsupervised clustering method or its simulation, for example, a SOM (Self-Organising Map).

3. A method for characterising paper as claimed in claim 1, characterised in that the feature describing the paper samples is a LBP or a bit pattern feature derived from it.

4. A method for characterising features of paper as claimed in claim 1, characterised in that according to the method, paper is in addition imaged and classified at different stages of its manufacture.

5. A method for characterising features of paper as claimed in claim 4, characterised in that the samples imaged at different stages of the manufacture are processed further by means of the unsupervised learning classifier in such a way that the classification will also concern the progressing of the manufacturing process.

6. A method as claimed in claim 5, characterised in that in addition to the image information, selected process parameters and/or measurement results are used as input.

7. A system for classifying paper using computer vision, characterised in that the system comprises imaging means, means for extracting the features describing paper quality from an image of the paper, and means for unsupervised learning classification into a space with a low-dimension space compared with the feature space.
